(19) United States
(12) Patent Application Publication
(10) Pub. No.: US 2026/0221284 A1
(43) Pub. Date: Jul. 30, 2026

(54) METHOD AND APPARATUS FOR PREDICTING CYBERSICKNESS IN VIRTUAL REALITY USING MULTIMODAL AND TIME-SERIES DATA

(71) Applicant: IUCF-HYU (INDUSTRY-UNIVERSITY COOPERATION FOUNDATION HANYANG UNIVERSITY), Seoul (KR)

(72) Inventors: Kyung Sik HAN, Seongnam-si (KR); Da Young Jeong, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 19/629,578

(22) Filed: Mar. 26, 2026

Related U.S. Application Data
(63) Continuation of application No. PCT/KR2024/014959, filed on Oct. 2, 2024.

(30) Foreign Application Priority Data
Oct. 5, 2023 (KR) 10-2023-0132842

(51) Int. Cl. G16H 50/20; A63F 13/213

(52) U.S. Cl. CPC G16H 50/20; A63F 13/213; A63F 2300/8082

(57) ABSTRACT

A method for predicting cybersickness in virtual reality using multimodal and time-series data includes: receiving, by a prediction apparatus, subject information associated with a subject; inputting, by the prediction apparatus, the subject information into a neural network model; and predicting, by the prediction apparatus, a cybersickness level of the subject based on an output of the neural network model. The subject information comprises eye movement data, head movement data, and physiological signal data. The neural network model is a transformer-based model configured to perform multi-head attention.

### CROSS-REFERENCE TO RELATED APPLICATIONS

This application is a Rule 53(b) Continuation of International Application No. PCT/KR2024/014959, filed on October 2, 2024, which claims priority to Korean Patent Application No. 10-2023-0132842, filed on October 5, 2023, in the Korean Intellectual Property Office, the entire disclosures of which are incorporated herein by reference in their entireties.

### BACKGROUND

### Technical Field

The technology described below relates to a method and apparatus for predicting cybersickness in virtual reality.

### Discussion of Related Art

Virtual reality (VR) is a technology for creating a specific environment (or situation, etc.) that is not real but is very similar to reality. Recently, as computer technology has developed, various contents using virtual reality have been introduced. For example, a technology has been developed in which a situation in which driving can be performed in virtual reality is created so that people can practice driving.

A user experiences cybersickness when experiencing virtual reality. Cybersickness means a motion sickness phenomenon felt by a user when enjoying a 3D game or video. Cybersickness is known to have various causes. Recently, various technologies for reducing cybersickness have been researched and developed.

In order to reduce cybersickness, it is necessary to analyze when cybersickness occurs upon viewing certain content. Accordingly, technologies for predicting cybersickness using artificial intelligence models based on artificial neural networks have been developed.

### SUMMARY OF THE INVENTION

In order to predict cybersickness using an artificial intelligence model, time-series data measured from various sensors is used. Conventionally, technologies for predicting cybersickness using artificial intelligence models based on artificial neural networks have used models based on convolutional neural networks (CNNs) and recurrent neural networks (RNNs). A model based on a convolutional neural network is specialized for learning data features, but has difficulty in processing time-series data. A model based on a recurrent neural network is specialized for processing time-series data, but has difficulty in learning data features.

The technology described below is intended to provide a method for predicting cybersickness in virtual reality using a neural network-based model capable of analyzing multimodal and time-series data.

### Technical Solution

A method for predicting cybersickness in virtual reality using multimodal and time-series data includes: receiving, by a prediction apparatus, subject information; inputting, by the prediction apparatus, the subject information into a neural network model; and predicting, by the prediction apparatus, a cybersickness level of a subject based on an output value of the neural network model.

The subject information includes eye movement data, head movement data, and physiological signal data.

The neural network model is a transformer-based model configured to perform multi-head attention.

By using the technology described below, a degree of cybersickness of a user experiencing virtual reality can be predicted and determined. In particular, the degree of cybersickness of the user can be predicted and determined based on various kinds of data. In addition, the degree of cybersickness of the user can be predicted and determined based on time-series data.

By using the technology described below, virtual reality content in which cybersickness occurs less can be produced.

### BRIEF DESCRIPTION OF DRAWINGS

FIG. 1 shows an overall process in which a prediction apparatus (100) performs a cybersickness prediction method.

FIG. 2 is a flowchart (200) of one embodiment of the cybersickness prediction method.

FIGS. 3 to 5 briefly show embodiments of a neural network model.

FIG. 6 shows a process of collecting subject information, preprocessing the subject information, and constructing a model based thereon.

FIGS. 7 to 11 show the structure of the constructed neural network model.

FIG. 12 shows a result of evaluating performance of the model.

FIGS. 13 to 15 show results of analyzing attention scores assigned to each data item by an attention MS-STTN model.

FIG. 16 is a configuration of one embodiment of a prediction apparatus (300).

### DETAILED DESCRIPTION

The technology described below may be modified in various ways and may have various embodiments. Specific embodiments of the technology described below may be described in the drawings of the specification. However, this is for describing the technology described below and is not intended to limit the technology described below to a specific embodiment. Accordingly, it should be understood that all changes, equivalents, and substitutes included within the spirit and technical scope of the technology described below are included in the technology described below.

In order to describe various components, terms such as first, second, A, and B may be used. However, the terms are used only to distinguish one component from other components, and are not intended to limit the corresponding components by the terms. For example, without departing from the scope of the technology described below, a first component may be named as a second component, and similarly, the second component may also be named as the first component. The term “and/or” includes a combination of a plurality of related listed items or any one of a plurality of related listed items.

In the terms used below, expressions in the singular should be understood as including expressions in the plural unless the context clearly indicates otherwise, and terms such as “comprises” should be understood as meaning that the stated features, numbers, steps, operations, components, parts, or combinations thereof are present, and not as excluding the presence or addition possibility of one or more other features, numbers, steps, operations, components, parts, or combinations thereof.

Before giving a detailed description of the drawings, it is intended to clarify that the division of components in this specification is merely made according to main functions performed by the respective components. That is, two or more components to be described below may be combined into one component, or one component may be provided by being divided into two or more components according to more subdivided functions. In addition, each of the components to be described below may additionally perform some or all of functions performed by another component, in addition to the main function for which the component is responsible, and of course, some functions among the main functions for which each component is responsible may be exclusively performed by another component.

In performing a method or an operation method, respective processes constituting the method may occur in an order different from the stated order unless a specific order is clearly stated from the context. That is, the respective processes may occur in the same order as the stated order, may be performed substantially simultaneously, or may be performed in the reverse order.

In the technology described below, a “module” may mean a component configured to perform a specific function. The module may be implemented in hardware, software, or a combination thereof. For example, the module may include program code executed by a processor, an algorithm, a software component, or an electronic circuit for executing the same.

Hereinafter, an overall process in which a prediction apparatus performs a method for predicting cybersickness in virtual reality using multimodal and time-series data (hereinafter, a cybersickness prediction method) will be described.

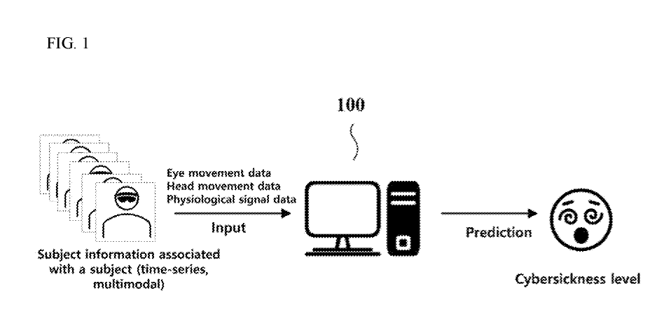

FIG. 1 shows an overall process in which a prediction apparatus (100) performs the cybersickness prediction method.

The prediction apparatus (100) may receive subject information associated with a subject. The prediction apparatus (100) may input the subject information associated with the subject into a neural network model. The prediction apparatus (100) may predict a cybersickness level of the subject based on an output of the neural network model. Further, the prediction apparatus (100) may preprocess the subject information associated with the subject.

The subject information associated with the subject may include eye movement data, head movement data, and physiological signal data. The neural network model may be a transformer-based model configured to perform multi-head attention.

We now describe the cybersickness prediction method in detail.

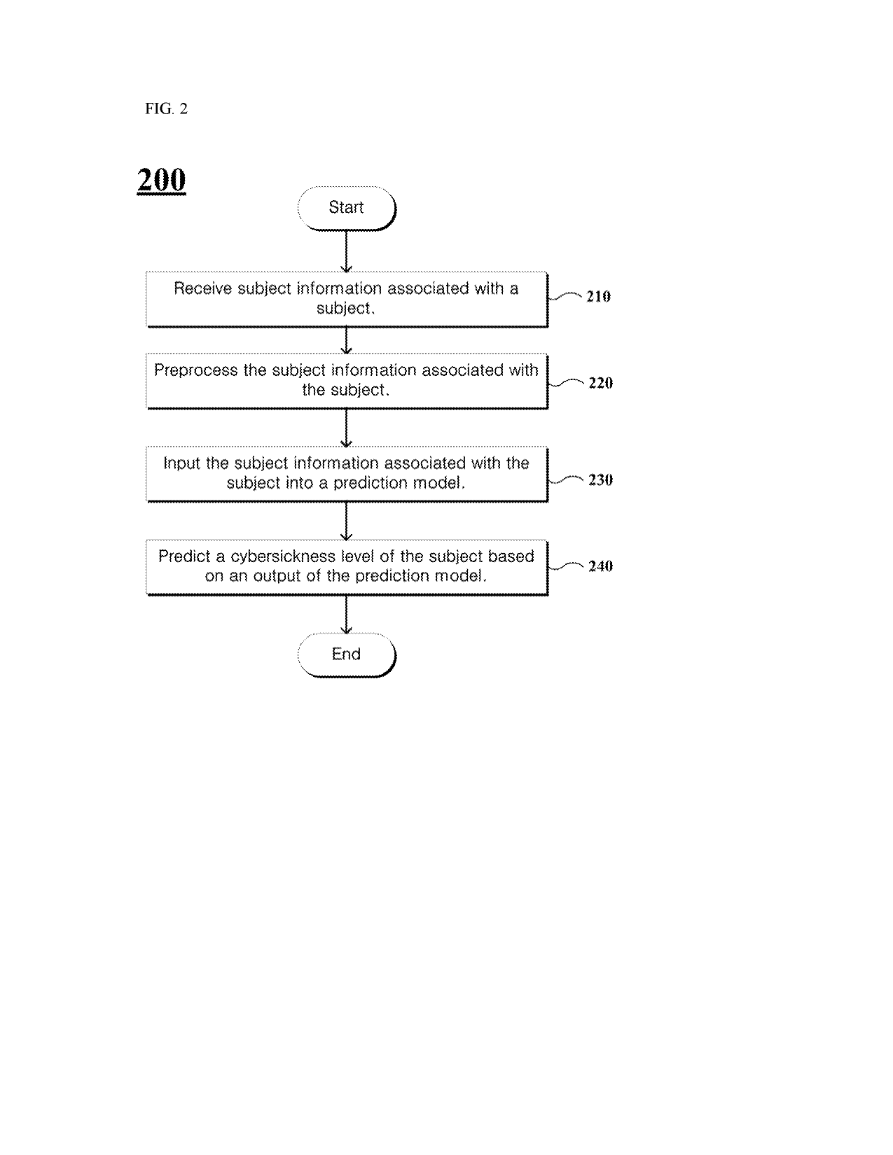

FIG. 2 is a flowchart (200) of one embodiment of the cybersickness prediction method.

The prediction apparatus may receive subject information associated with a subject (210).

The subject information may include information required to predict cybersickness.

The subject information may be information obtained in a process in which the subject wears a virtual reality device. For example, the subject information may be information measured in a state in which the subject wearing a VR HMD (head-mounted display) experiences virtual reality.

The subject information may be information measured according to a flow of time. That is, the subject information may include time-series data. For example, the subject information may be information measured from the subject wearing the virtual reality device for 45 seconds.

The subject information may be information measured at a predetermined period. For example, the subject information may be information measured at a period of 60 times per second (60 Hz).

The subject information may include eye movement data, head movement data, and physiological signal data.

The eye movement data may include 23 types of data.

The eye movement data may include at least one of X, Y, and Z coordinates of a gaze direction (gaze direction (x, y, z)) of each of the left and right eyes, X, Y, and Z coordinates of a gaze origin (gaze origin (x, y, z)) of each of the left and right eyes, a pupil diameter of each of the left and right eyes, X and Y coordinates of a pupil position (pupil position (x, y)) of each of the left and right eyes, eye openness of each of the left and right eyes, and X, Y, and Z coordinates of a gaze direction for both eyes combined (gaze direction with both eyes integrated (x, y, z)).

The head movement data may include 6 types of data.

The head movement data may include at least one of X, Y, and Z coordinates of a head position (head position (x, y, z)) and roll, pitch, and yaw of head rotation (head rotation (roll, pitch, yaw)).

The physiological signal data may include 3 types of data.

The physiological signal data may include at least one of electrodermal activity (EDA), blood volume pulse (BVP), and skin temperature.

The prediction apparatus may preprocess the subject information (220).

The preprocessing may include at least one of removing duplicated data from data included in the subject information, normalizing the subject information, and performing a Fourier transform on the subject information.

The preprocessing may include removing duplicated values. In one embodiment, in order to remove a data redundancy phenomenon occurring when data is collected, the preprocessing may include removing data in which values are successively duplicated.

The preprocessing may include normalizing the subject information. In one embodiment, the preprocessing may include normalizing the subject information to values between 0 and 1.

The preprocessing may include performing a Fourier transform on the subject information. Further, the preprocessing may include performing a short-time Fourier transform (STFT) on the subject information. In one embodiment, the preprocessing may include performing a short-time Fourier transform on the subject information having a total length of 45 seconds at intervals of 15 seconds.

The prediction apparatus may input the subject information into a neural network model (230).

The neural network model may be a model for predicting a cybersickness level of the subject based on the subject information.

The neural network model may be an artificial neural network-based model. The neural network model may be a transformer-based model configured to perform multi-head attention. Specifically, the neural network model may have a structure of a Vision Transformer.

The neural network model may embed the subject information. For this purpose, the neural network model may use a Linear Projection layer. Alternatively, the neural network model may include a linear patch embedding layer. The linear patch embedding layer may map input data to a uniform vector size. Through this, a uniform vector size D may be used in all layers of the transformer.

The neural network model may include a positional embedding layer that reflects positional information in the received subject information. The positional embedding layer is used to reflect positional information of each piece of data because it is difficult for the transformer to know an order of position vectors.

The neural network model may add a learnable class token (CLS) before passing through the positional embedding layer. The CLS may serve as a representation vector having important features for data in predicting cybersickness after passing through the transformer.

The neural network model may be a model trained to identify characteristics of each modality (eye movement, head movement, and physiological signals).

The neural network model may be of three types. A first neural network model may include a Temporal Transformer Encoder. A second neural network model may sequentially include a Spatial Transformer Encoder and a Temporal Transformer Encoder. A third neural network model may include a first module, a second module, and a third module, each sequentially including a Spatial Transformer Encoder and a Temporal Transformer Encoder.

A detailed description of the neural network model is provided below.

The prediction apparatus may predict a cybersickness level of the subject based on an output of the neural network model (240).

The cybersickness level may be divided into a plurality of stages. In one embodiment, the cybersickness level may be divided into four stages according to a degree of cybersickness (0: no dizziness at all. 1: slight dizziness 2: some dizziness 3: severe dizziness).

The neural network model is described in detail below.

Hereinafter, the neural network model will be described in detail below.

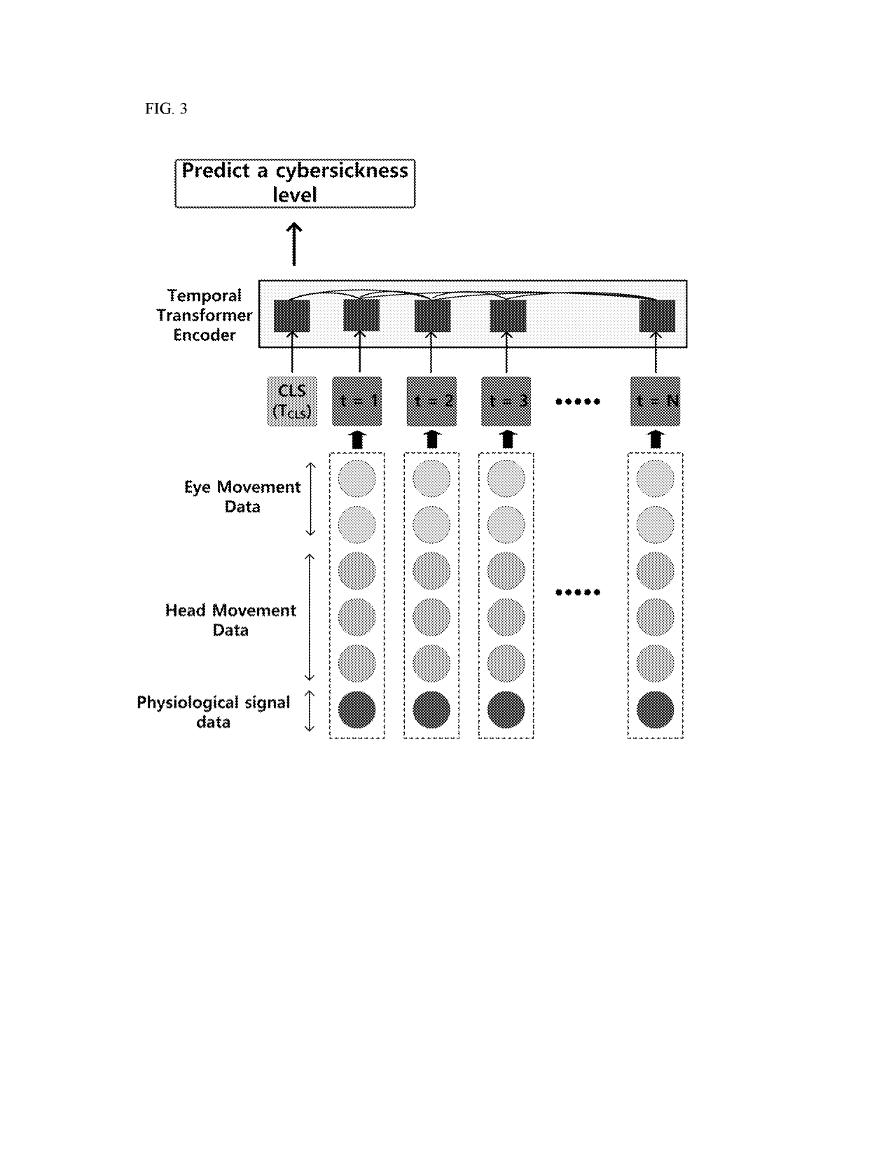

FIGS. 3 to 5 briefly show embodiments of the neural network model.

As shown in FIG. 3, the neural network model may include a Temporal Transformer Encoder.

The Temporal Transformer Encoder may perform multi-head attention based on relationships between data at different time points (t). The Temporal Transformer Encoder may perform multi-head attention based on relationships between values obtained by embedding data at different time points.

Specifically, the Temporal Transformer Encoder may analyze relationships between values obtained by embedding data at different time points, calculate temporal attention scores, and assign attention to each of the values obtained by embedding data at different time points based on the temporal attention scores.

The values obtained by embedding the data may be a single vector value generated by combining eye movement data, head movement data, and physiological signal data measured at the same time point. For example, the values obtained by embedding the data may be a single vector value generated by passing 23 types of eye movement data, 6 types of head movement data, and 3 types of physiological signals measured at 1 second through a linear patch embedding layer.

A Class Token (CLS) may be input to the Temporal Transformer Encoder. Specifically, TCLS may be input to the Temporal Transformer Encoder. TCLS may be a representation vector having an important role in predicting cybersickness after passing through the Temporal Transformer.

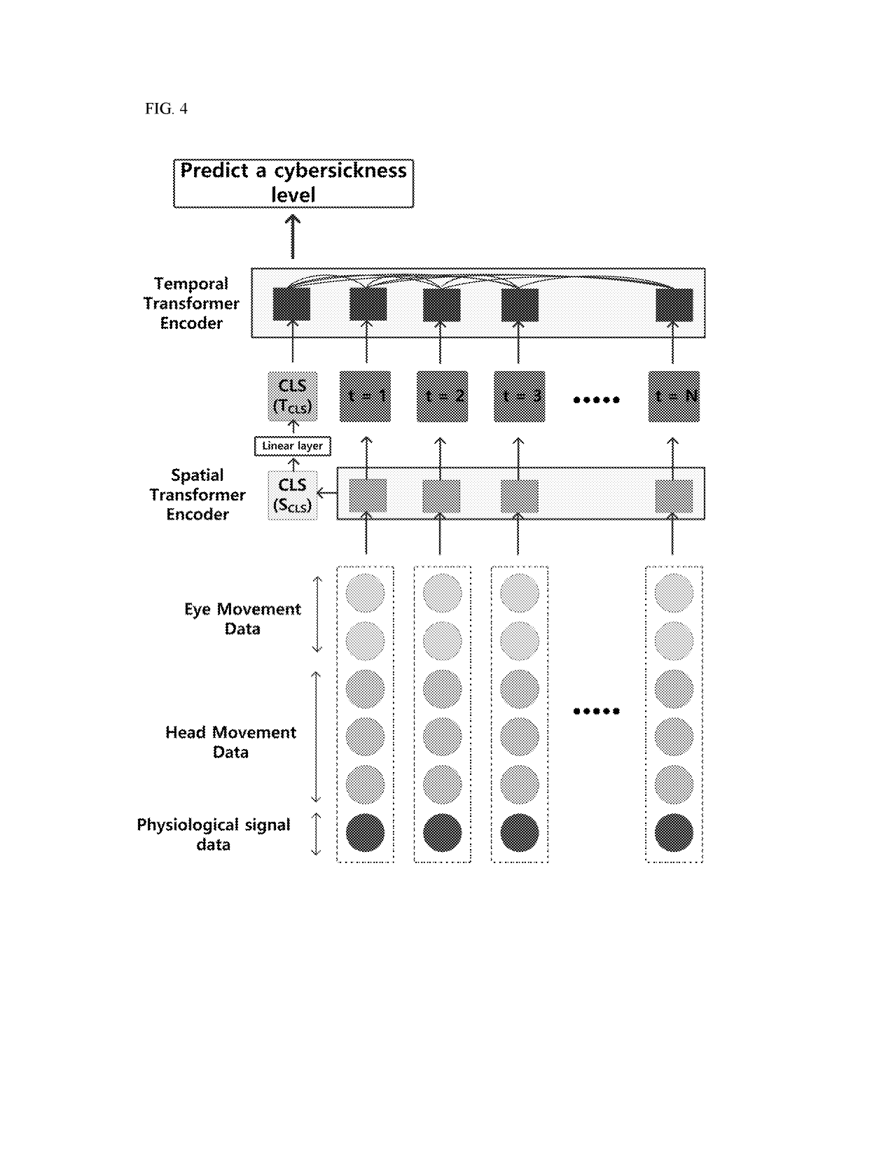

As shown in FIG. 4, the neural network model may sequentially include a Spatial Transformer Encoder and a Temporal Transformer Encoder.

The Spatial Transformer Encoder may perform multi-head attention based on relationships between data included at one time point.

The Spatial Transformer Encoder may perform multi-head attention based on relationships between values obtained by embedding data included at one time point.

Specifically, the Spatial Transformer Encoder may analyze relationships between values obtained by embedding data included at one time point, calculate spatial attention scores, and assign attention to each of the values obtained by embedding data included at one time point based on the spatial attention scores.

The Spatial Transformer Encoder may generate an embedding value for one time point in which spatial information is reflected by concatenating values obtained by embedding data included at one time point to which spatial attention is assigned.

The Spatial Transformer Encoder may receive data at a plurality of time points and generate data embedding values in which spatial information of each time point is reflected. In one embodiment, the Spatial Transformer Encoder may receive data at 45 time points measured every 1 second for 45 seconds and generate 45 data embedding values in which spatial information of each time point is reflected.

The Temporal Transformer Encoder may receive an output of the Spatial Transformer Encoder and perform multi-head attention based on relationships between data at different time points. That is, values obtained by embedding data input to the Temporal Transformer Encoder may be embedding values for one time point in which spatial information output from the Spatial Transformer Encoder is reflected.

The Spatial Transformer Encoder may extract a Class Token (CLS). Specifically, the Spatial Transformer Encoder may extract SCLS. The SCLS extracted by the Spatial Transformer Encoder may pass through a Linear Layer and then be matched to a dimension of TCLS input to the Temporal Transformer. Accordingly, TCLS may reflect both spatial characteristics and temporal characteristics.

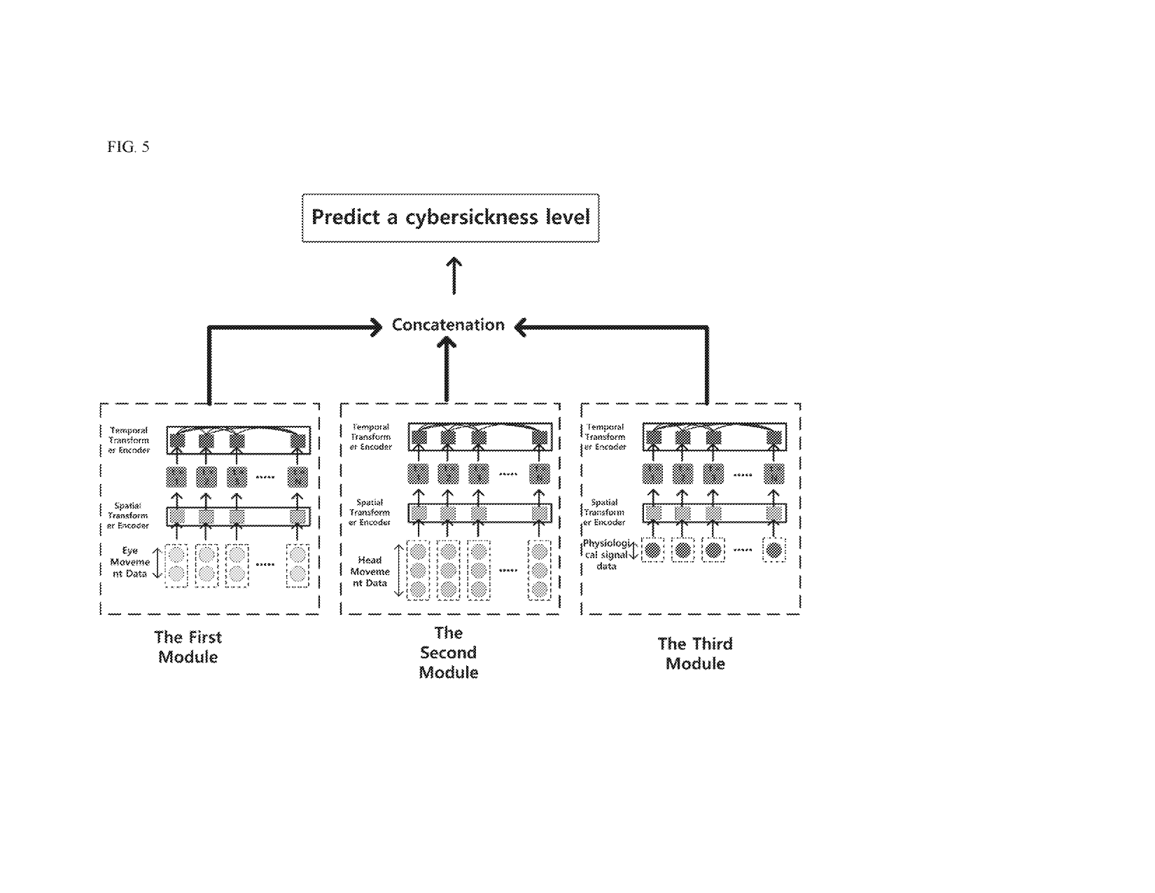

As shown in FIG. 5, the neural network model may include a first module, a second module, and a third module, each sequentially including a Spatial Transformer Encoder and a Temporal Transformer Encoder. That is, each of the first module, the second module, and the third module may sequentially include a Spatial Transformer Encoder and a Temporal Transformer Encoder.

The neural network model may calculate information required to predict a cybersickness level after concatenating outputs of the first module, the second module, and the third module.

The first module may receive eye movement data among the subject information. The second module may receive head movement data among the subject information. The third module may receive physiological signal data among the subject information.

By using the Spatial Transformer Encoder and the Temporal Transformer Encoder, the first module, the second module, and the third module may analyze spatial characteristics and temporal characteristics of eye movement data, head movement data, and physiological signal data, respectively, and then generate information required to predict cybersickness. In other words, each of the first module, the second module, and the third module may perform operations of the model described in FIG. 4.

Hereinafter, after actually constructing the neural network model used in the disclosure, experimental results of predicting cybersickness using the constructed model will be described.

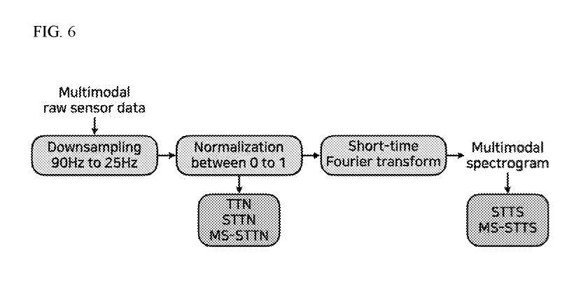

FIG. 6 shows a process of collecting subject information, preprocessing the subject information, and constructing a model based thereon.

The subject information was collected from 45 subjects who watched 20 pieces of 360 VR content.

The subject information includes multimodal sensor data. The subject information includes eye movement data (e1, e2, ... e23), head movement data (h1, h2, h3, ... h6), and physiological signal data (p1, p2, p3). The eye movement data and the head movement data were measured from a VR head-mounted display (HMD). The physiological signal data were measured from a physiological signal device (Empatica E4 wristband).

The eye movement data used in the experiment include 23 types of data. The eye movement data used in the experiment include X, Y, and Z coordinates of a gaze direction (gaze direction (x, y, z)) of each of the left and right eyes, X, Y, and Z coordinates of a gaze origin (gaze origin (x, y, z)) of each of the left and right eyes, a pupil diameter of each of the left and right eyes, X and Y coordinates of a pupil position (pupil position (x, y)) of each of the left and right eyes, eye openness of each of the left and right eyes, and X, Y, and Z coordinates of a gaze direction for both eyes combined (gaze direction with both eyes integrated (x, y, z)).

The head movement data used in the experiment include 6 types of data. The head movement data used in the experiment include X, Y, and Z coordinates of a head position (head position (x, y, z)) and roll, pitch, and yaw of head rotation (head rotation (roll, pitch, yaw)).

The physiological signal data used in the experiment include 3 types of data. The physiological signal data used in the experiment include electrodermal activity (EDA), blood volume pulse (BVP), and skin temperature.

The subject information was measured at a measurement period of 90 times per second (90 Hz).

The cybersickness level of the subject was analyzed every 15 seconds. The cybersickness level was measured from a Fast Motion Sickness (FMS) response. FMS is used to quantitatively measure the cybersickness level.

The measured subject information was preprocessed.

First, in order to solve a data duplication phenomenon generated when sensor data are collected, successively duplicated values were removed. Through this, the measured subject information was downsampled from 90 Hz to 25 Hz.

Thereafter, the subject information was normalized to values between 0 and 1. As described below, the downsampled and normalized subject information was used to construct TTN, STTN, and MS-STTN models.

Thereafter, the subject information was subjected to a short-time Fourier transform (STFT) and converted into multimodal spectrogram data. As described below, the downsampled, normalized, and short-time Fourier-transformed subject information was used to construct STTS and MS-STTS models.

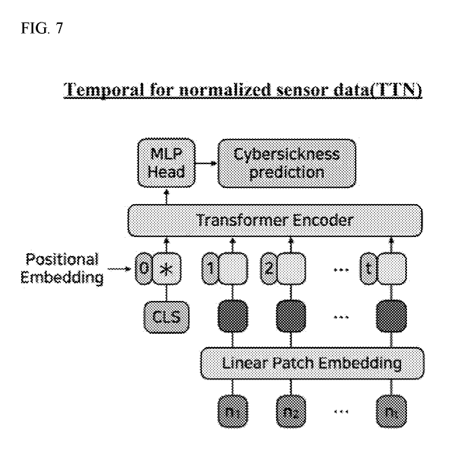

FIGS. 7 to 11 show structures of the constructed neural network models.

The number of constructed neural network models is five in total.

As shown in FIG. 7, the first neural network model is a model including a Temporal Transformer Encoder. This may be referred to as temporal transformer for normalized sensor data (TTN).

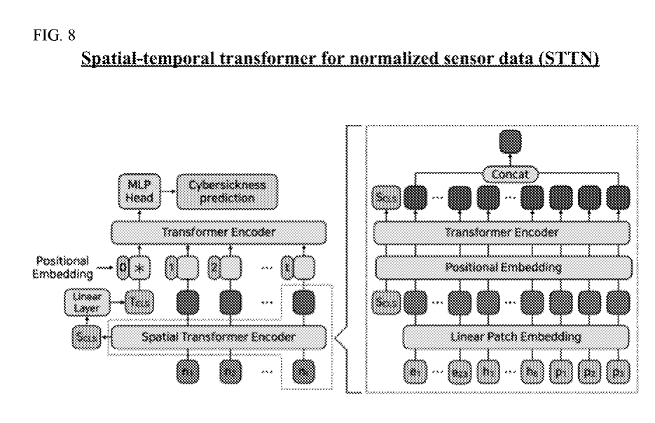

As shown in FIG. 8, the second neural network model is a model including a Spatial Transformer Encoder. This may be referred to as a Spatial Transformer Encoder (STE).

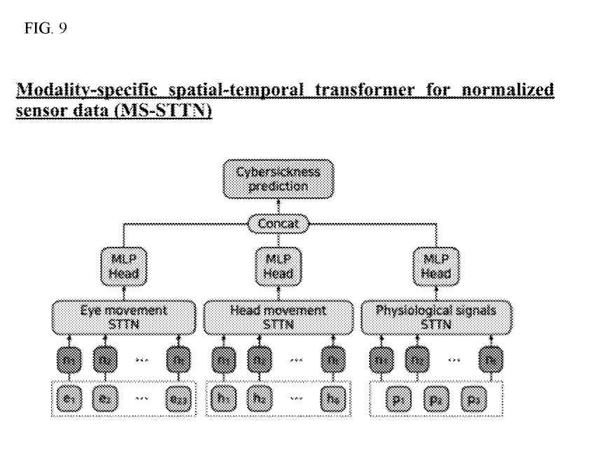

As shown in FIG. 9, the third neural network model may include a first module, a second module, and a third module, each sequentially including a Spatial Transformer Encoder and a Temporal Transformer Encoder. This may be referred to as modality-specific spatial-temporal transformer for normalized sensor data (MS-STTN).

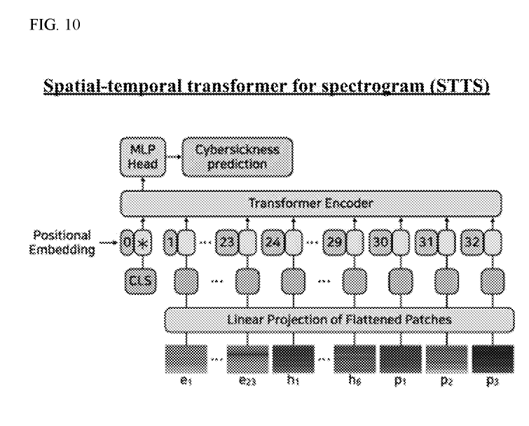

As shown in FIG. 10, the fourth neural network model is obtained by preprocessing the subject information by STFT and then inputting the preprocessed subject information into the above-described TTN model. This may be referred to as spatial-temporal transformer for spectrogram (STTS).

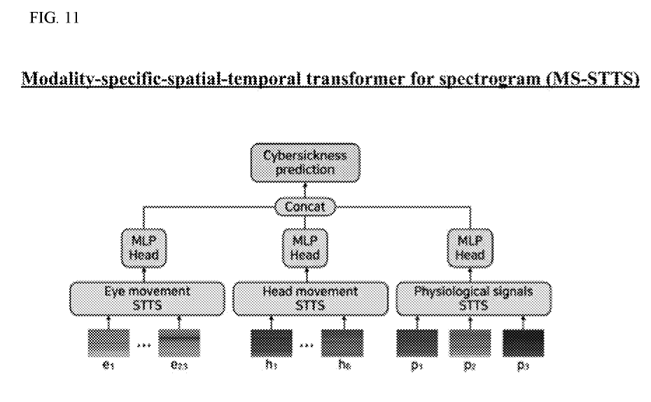

As shown in FIG. 11, the fifth neural network model is obtained by preprocessing the subject information by STFT and then inputting the preprocessed subject information into the above-described MS-STTN model. This may be referred to as modality-specific spatial-temporal transformer for spectrogram (MS-STTS).

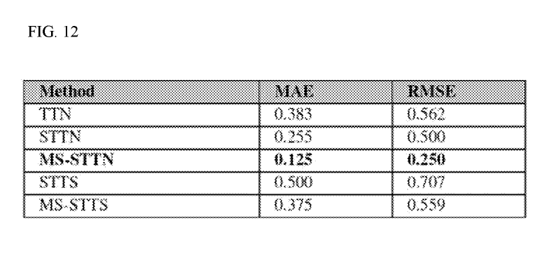

FIG. 12 shows results of evaluating performance of the model.

Experimental results confirm that MS-STTN has the highest performance. Through this, it can be seen that using normalized data, applying transformer encoders independent for each modality to perform learning, and then fusing the same to predict cybersickness is effective.

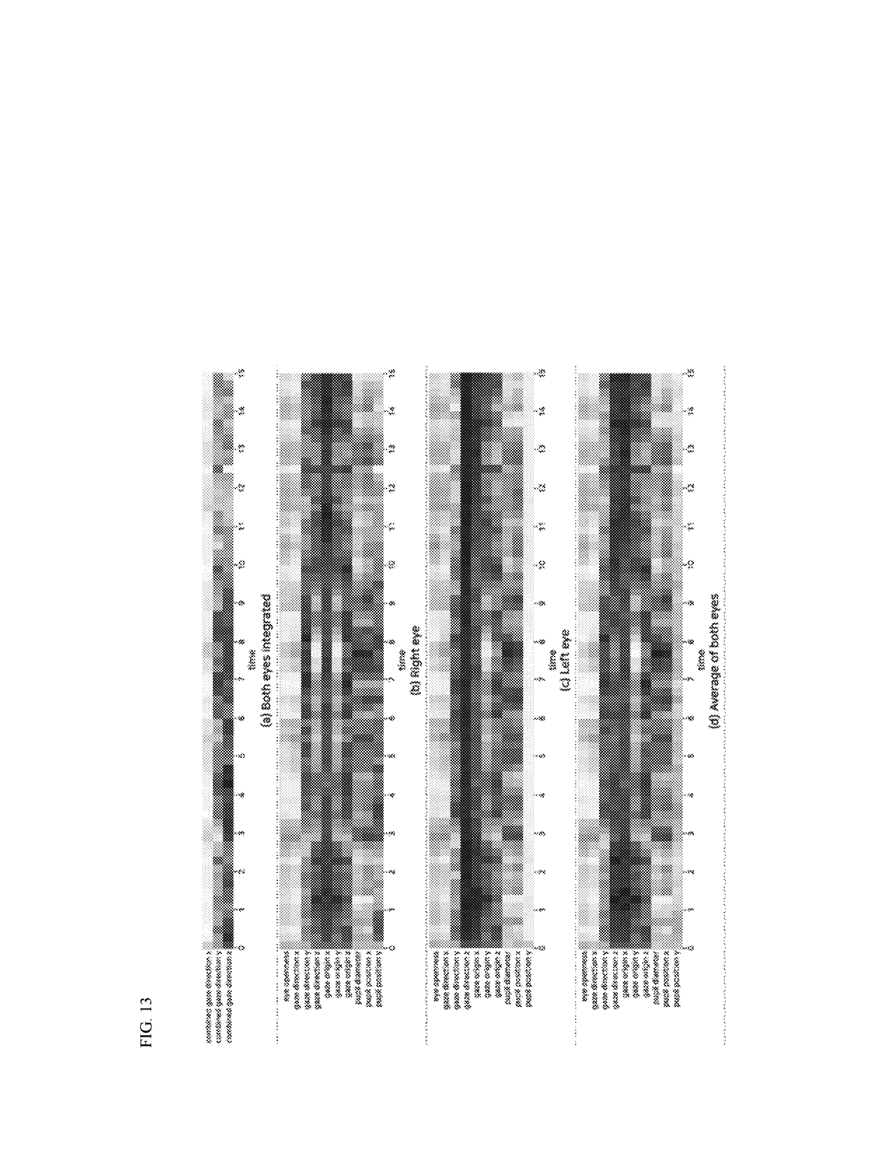

FIGS. 13 to 15 show results of analyzing attention scores assigned to each data item by the MS-STTN model used for attention-score analysis

FIG. 13 shows attention scores assigned to eye movement data. FIG. 13(a) shows attention scores assigned to data for both eyes combined. FIG. 13(b) shows attention scores assigned to data of the right eye. FIG. 13(c) shows attention scores assigned to data of the left eye. FIG. 13(d) shows an average of attention scores assigned to the right eye and the left eye.

Among the data for both eyes combined, the Z coordinate of the gaze direction shows the highest importance. It can be confirmed that, among the data for both eyes combined, the Y coordinate of the gaze direction has a large effect on predicting cybersickness in a middle phase.

It can be confirmed that the X coordinate and the Y coordinate of the gaze origin of the right eye show high importance in most phases. On the other hand, it can be confirmed that the X coordinate of the gaze origin of the left eye and the Z coordinate of the gaze direction of the left eye show high importance in most phases.

It can be confirmed that the Y coordinate of the gaze direction, the pupil diameter, and the X coordinate of the pupil position have an effect in a middle phase.

It can be confirmed that the X coordinate of the gaze direction, eye openness, and the X-axis coordinate of the gaze direction for both eyes combined have a low effect in all phases.

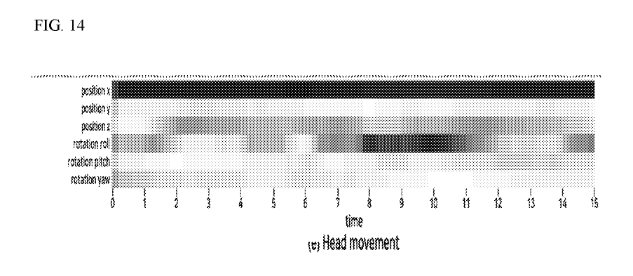

FIG. 14 shows attention scores assigned to head movement data.

It can be confirmed that the X coordinate of the head position has the greatest effect on cybersickness prediction in all phases. It can be confirmed that the roll of the head rotation has a large effect in middle and last phases. This may be because the roll of the head rotation means rotation about the x-axis, such as nodding the head forward or tilting the head backward. In many cases, VR videos are 360 degrees, and the test subject shows many head movements in order to explore the surroundings while experiencing virtual reality content. Such head movements may cause postural instability and display delay, thereby causing cybersickness.

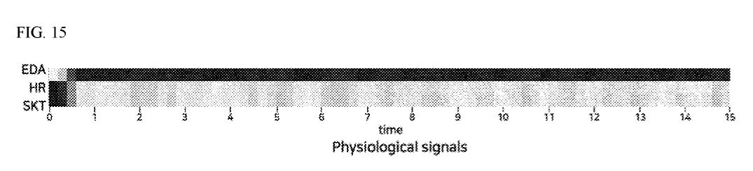

FIG. 15 shows attention scores assigned to physiological signal data.

It can be confirmed that electrodermal activity has the highest effect on cybersickness in all phases.

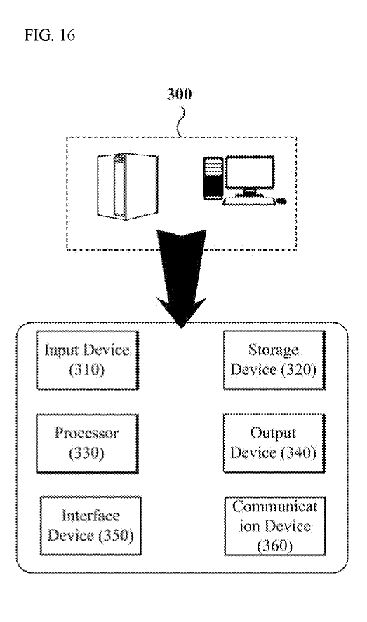

A prediction apparatus will be described below with reference to FIG. 16.

FIG. 16 is a configuration of one embodiment of a prediction apparatus (300).

The prediction apparatus (300) may correspond to the prediction apparatus (100) described in FIG. 1. That is, the prediction apparatus (300) may be an apparatus for performing the above-described cybersickness prediction method.

The prediction apparatus (300) may be physically implemented in various forms. For example, the prediction apparatus (300) may have a form of a PC, a notebook computer, a smart device, a server, or a chipset dedicated to data processing.

The prediction apparatus (300) may include an input device (310), a storage device (320), a processor (330), an output device (340), an interface device (350), and a communication device (360).

The input device (310) may include an interface device (keyboard, mouse, touch screen, or the like) for receiving a predetermined command or data. The input device (310) may include a configuration for receiving information through a separate storage device (USB, CD, hard disk, or the like). The input device (310) may receive input data through a separate measuring device, or may receive input data through a separate DB. The input device (310) may also receive data through wired or wireless communication through the communication device (360). The input device (310) may receive information required to perform the above-described cybersickness prediction method. The input device (310) may receive a neural network model required to perform the above-described cybersickness prediction method.

The input device (310) may receive subject information associated with a subject. The input device (310) may receive a neural network model.

The storage device (320) may be a device that stores predetermined information. The storage device (320) may store information input through the input device (310). The storage device (320) may store information generated in a process in which the processor (330) performs an operation. That is, the storage device (320) may include a memory.

The storage device (320) may store information required to perform the above-described cybersickness prediction method. The storage device (320) may store a neural network model required to perform the above-described cybersickness prediction method. The storage device (320) may store subject information associated with a subject. The storage device (320) may store the neural network model.

The processor (330) may be a device such as a processor, an AP, or a chip in which a program is embedded, the device processing data and processing a predetermined operation. The processor (330) may generate a control signal for controlling the prediction apparatus (300). The processor (330) may generate a control signal for controlling the input device (310), the storage device (320), the output device (340), the interface device (350), and the communication device (360) included in the prediction apparatus (300).

The processor (330) may perform an operation required to perform the above-described cybersickness prediction method. The processor (330) may input subject information associated with a subject into the neural network model. The processor (330) may predict a cybersickness level of the subject based on an output of the neural network model. The processor (330) may preprocess the subject information associated with the subject.

The output device (340) may be a device that outputs predetermined information. The output device (340) may output an interface required for a data process, input data, an analysis result, and the like. The output device (340) may be physically implemented in various forms such as a display, a device for outputting a document, and a speaker. The output device (340) may output information stored in the storage device (340). The output device (340) may output information generated in a process in which the processor (330) performs an operation. The output device (340) may output a result calculated by the processor (330).

The interface device (350) may be a device for receiving a predetermined command and data from the outside. The interface device (350) may receive a control signal for controlling the prediction apparatus (300). The interface device (350) may output a result analyzed by the prediction apparatus (300). The interface device (350) may receive, from a physically connected input device or an external storage device, information and a neural network model required to perform the above-described cybersickness prediction method.

The communication device (360) may mean a configuration for receiving and transmitting predetermined information through a wired or wireless network. The communication device (360) may perform network communication such as Wi-Fi (Wireless Fidelity), Wi-Fi Direct, Bluetooth, UWB (Ultra-Wide Band), NFC (Near Field Communication), USB (Universal Serial Bus), HDMI (High Definition Multimedia Interface), or LAN (Local Area Network). The communication device (360) may receive a control signal required to control the prediction apparatus (300). The communication device (360) may transmit a result analyzed by the prediction apparatus (300). The communication device (360) may receive information required to perform the above-described cybersickness prediction method. The communication device (360) may receive a neural network model required to perform the above-described cybersickness prediction method.

The above-described cybersickness prediction method may be implemented as a program (or application) including an executable algorithm that can be executed in a computer.

The program may be stored and provided in a transitory or non-transitory computer readable medium.

The transitory computer readable medium means various RAMs such as static RAM (SRAM), dynamic RAM (DRAM), synchronous DRAM (SDRAM), double data rate SDRAM (DDR SDRAM), enhanced SDRAM (ESDRAM), Synclink DRAM (SLDRAM), and direct Rambus RAM (DRRAM).

The non-transitory computer readable medium means not a medium that stores data for a short moment, such as a register, a cache, or a memory, but a medium that stores data semi-permanently and is readable by a device. Specifically, the various applications or programs described above may be stored and provided in a non-transitory computer readable medium such as a CD, a DVD, a hard disk, a Blu-ray disk, a USB, a memory card, ROM (read-only memory), PROM (programmable read-only memory), EPROM (erasable PROM), EEPROM (electrically erasable PROM), or a flash memory.

The present embodiment and the drawings attached to this specification merely clearly show a part of the technical spirit included in the above-described technology, and it will be apparent that all modifications and specific embodiments that can be easily inferred by a person skilled in the art within the scope of the technical spirit included in the specification and drawings of the above-described technology are included in the scope of rights of the above-described technology.

What is claimed is:

1. A method for predicting cybersickness in virtual reality using multimodal and time-series data, the method comprising:
receiving, by a prediction apparatus, subject information associated with a subject;
inputting, by the prediction apparatus, the subject information into a neural network model; and
predicting, by the prediction apparatus, a cybersickness level of the subject based on an output of the neural network model,
wherein the subject information comprises eye movement data, head movement data, and physiological signal data, and
wherein the neural network model is a transformer-based model configured to perform multi-head attention.

2. The method of claim 1,
wherein the eye movement data comprises at least one of X, Y, and Z coordinates of a gaze direction (gaze direction (x, y, z)) of each of the left and right eyes, X, Y, and Z coordinates of a gaze origin (gaze origin (x, y, z)) of each of the left and right eyes, a pupil diameter of each of the left and right eyes, X and Y coordinates of a pupil position (pupil position (x, y)) of each of the left and right eyes, eye openness of each of the left and right eyes, and X, Y, and Z coordinates of a gaze direction for both eyes combined (gaze direction with both eyes integrated (x, y, z)).

3. The method of claim 1,
wherein the head movement data comprises at least one of X, Y, and Z coordinates of a head position (head position (x, y, z)) and roll, pitch, and yaw of head rotation (head rotation (roll, pitch, yaw)).

4. The method of claim 1,
wherein the physiological signal data comprises at least one of electrodermal activity (EDA), blood volume pulse (BVP), and skin temperature.

5. The method of claim 1, further comprising preprocessing, by the prediction apparatus, the subject information,
wherein the preprocessing comprises at least one of removing duplicated data from data included in the subject information, normalizing the subject information, and performing a Fourier transform on the subject information.

6. The method of claim 1,
wherein the neural network model is a Vision Transformer-based model.

7. The method of claim 1,
wherein the neural network model comprises a Temporal Transformer Encoder, and
wherein the Temporal Transformer Encoder performs multi-head attention based on relationships between data at different time points.

8. The method of claim 1,
wherein the neural network model sequentially comprises a Spatial Transformer Encoder and a Temporal Transformer Encoder,
wherein the Spatial Transformer Encoder performs multi-head attention based on relationships between data included at one time point, and
wherein the Temporal Transformer Encoder receives an output of the Spatial Transformer Encoder as input and performs multi-head attention based on relationships between data at different time points.

9. The method of claim 1,
wherein the neural network model comprises a first module, a second module, and a third module, each sequentially comprising a Spatial Transformer Encoder and a Temporal Transformer Encoder,
wherein the Spatial Transformer Encoder performs multi-head attention based on relationships between data included at one time point,
wherein the Temporal Transformer Encoder receives an output of the Spatial Transformer Encoder as input and performs multi-head attention based on relationships between data at different time points,
wherein the first module receives eye movement data among the subject information,
wherein the second module receives head movement data among the subject information,
wherein the third module receives physiological signal data among the subject information, and
wherein the neural network model predicts a cybersickness level based on output values of the first module, the second module, and the third module.

10. A prediction apparatus for predicting cybersickness in virtual reality using multimodal and time-series data, comprising:
an input device configured to receive subject information associated with a subject;;
a processor configured to input the subject information into a neural network model and predict a cybersickness level of a subject based on an output of the neural network model; and
a storage device configured to store the subject information and the neural network model,
wherein the subject information comprises eye movement data, head movement data, and physiological signal data, and
wherein the neural network model is a transformer-based model configured to perform multi-head attention.

11. The prediction apparatus of claim 10,
wherein the processor preprocesses the subject information, and
wherein the preprocessing comprises at least one of removing duplicated data from data included in the subject information, normalizing the subject information, and performing a Fourier transform on the subject information.

12. The prediction apparatus of claim 10,
wherein the neural network model comprises a Temporal Transformer Encoder, and
wherein the Temporal Transformer Encoder performs multi-head attention based on relationships between data at different time points.

13. The prediction apparatus of claim 10,
wherein the neural network model sequentially comprises a Spatial Transformer Encoder and a Temporal Transformer Encoder,
wherein the Spatial Transformer Encoder performs multi-head attention based on relationships between data included at one time point, and
wherein the Temporal Transformer Encoder receives an output of the Spatial Transformer Encoder as input and performs multi-head attention based on relationships between data at different time points.

14. The prediction apparatus of claim 10,
wherein the neural network model comprises a first module, a second module, and a third module, each sequentially comprising a Spatial Transformer Encoder and a Temporal Transformer Encoder,
wherein the Spatial Transformer Encoder performs multi-head attention based on relationships between data included at one time point,
wherein the Temporal Transformer Encoder receives an output of the Spatial Transformer Encoder as input and performs multi-head attention based on relationships between data at different time points,
wherein the first module receives eye movement data among the subject information,
wherein the second module receives head movement data among the subject information,
wherein the third module receives physiological signal data among the subject information, and
wherein the neural network model predicts a cybersickness level based on output values of the first module, the second module, and the third module.

15. A non-transitory computer-readable medium storing instructions that, when executed by one or more processors, cause the one or more processors to perform the method of claim 1.
